(19) United States
(12) Patent Application Publication
(10) Pub. No.: US 2004/0019066 A1
(43) Pub. Date: Jan. 29, 2004

(54) Methionine salvage pathway in bacillus

(75) Inventors: Antoine Danchin, Pokfulam (HK); Agnieszka Sekowska, Pokfulam (HK)

Correspondence Address: OBLON, SPIVAK, MCCLELLAND, MAIER & NEUSTADT, P.C., 1940 DUKE STREET, ALEXANDRIA, VA, 22314, US

(73) Assignee: INSTITUT PASTEUR, Paris Cedex (FR)

(21) Appl. No.: 10/429,872

(22) Filed: May 6, 2003

Related U.S. Application Data
(60) Provisional application No. 60/377,622, filed on May 6, 2002.

(51) Int. Cl. A61K 31/522

(52) U.S. Cl. 514/263.38

(57) ABSTRACT

The present invention relates to pathways for the synthesis and recycling of methylthioribose (MTR), applications in the fight against plant and vertebrate pathogens (including parasites and their vectors), application for the production of fine chemicals, and in fermentation industry. The present invention also relates to the identification of new drug targets in previously unknown metabolic pathways in living organisms, in particular in bacteria, yeasts, mold, parasites and plants.

### RELATED APPLICATIONS

This application claims benefit under 35 U.S.C. §119(a) to Provisional Application Serial No. 60/377,622, filed on May 6, 2002, and incorporated herein by reference.

### BACKGROUND OF THE INVENTION

1. Field of the Invention

The present invention relates to pathways for the synthesis and recycling of methylthioribose (MTR), applications in the fight against plant and vertebrate pathogens (including parasites and their vectors), application for the production of fine chemicals, and in fermentation industry. The present invention also relates to the identification of new drug targets in previously unknown metabolic pathways in living organisms, in particular in bacteria, yeasts, mold, parasites and plants.

2. Description of the Background

Polyamine synthesis produces methylthioadenosine, which has to be disposed of. The cell recycles it into methionine through methylthioribose (MTR). Very little was known about MTR recycling for methionine salvage in Bacilli, particularly Bacillus subtilis.

The fate of methylthioribose (MTR), the end-product of spermidine and spermine metabolism, as well as of ethylene biosynthesis has not yet been fully explored in most organisms. In Escherichia coli this molecule is excreted in the medium [1] while in Klebsiella pneumoniae it constitutes the methionine salvage pathway, being metabolized back into methionine [2, 3]. In eukaryotic parasites it is also recycled into methionine, presumably through a pathway similar to that in K. pneumnoniae [4]. In Bacillus subtilis we found that MTR is an excellent sulfur source [5] and we unraveled some of the steps involved in its metabolism, which starts from phosphorylation of MTR, mediated by the MtnK protein [6].

It has been shown previously that the ykrW gene has links with sulfur metabolism. Indeed, Henkin and co-workers found that the corresponding coding sequence (CDS) was preceded by a S-box typical of sulfur metabolism genes in B. subtilis [7] and Hanson and Tabita found that two classes of enzymes similar to ribulose phosphate carboxylase/oxygenases (Rubisco) were associated with sulfur metabolism [8].

This raised interesting questions about the origin of this pathway. In particular the YkrW gene origin could have been early in evolution, or resulting from lateral transfer from plants to bacilli.

### SUMMARY OF THE INVENTION

We demonstrate here that proteins YkrUWXYZ are needed for MTR recycling into methionine in B. subtilis, while YkrV, an aminotransferase, is probably more specific of methionine transamination, but is dispensable in the present conditions because of the present of a variety of izozymes (up to nine amino acid transaminases are present in B. subtilis).

Using in silico genome analysis and transposon mutagenesis in B. subtilis we have experimentally uncovered the major steps of the dioxygen-dependent methionine salvage pathyway, which, although similar to that found in Klebsiella pneumoniae, recruited for its implementation some entirely different proteins. The promoters of the genes have been identified by primer extension, and gene expression was analyzed by Northern blotting and lacZ reporter gene expression. Among the most remarkable discoveries in this pathway is the role of an analog of ribulose diphosphate carboxylase (Rubisco, the plant enzyme used in the Dalvin cycle which recovers carbon dioxide from the atmosphere) as a major step in MTR recycling.

Thus, a complete methionine salvage pathway exists in B. subtilis. This pathway is chemically similar to that in K. pneumoniae, but recruited different proteins to this purpose. In particular, a paralogue or Rubisco, MtnW, is used at one of the steps in the pathway. A major observation is that in the absence of MtnW MTR becomes extremely toxic to the cell, opening an unexpected target for new antimicrobial drugs. In addition to methionine salvage, this pathway protects B. subtilis against dioxygen produced by its natural biotope, the surface of leaves (phylloplane).

As described herein, we discovered that the natural product MTR is toxic in mtnU or mtnW mutants, provided mtnY is functional this demonstrated that the immediate product of MtnY action (5-thiomethylribulose-1-phosphate) is toxic to the cells. This molecule is, therefore, a lead for new drugs. Mimics and analogs would inhibit cell multiplication. The downstream product 2,3-diketo-5-methylthio-phosphopentane, which is highly related to 5-thiomethylribulose-1-phosphate may be suitable for this purpose. This is also an important discovery that should be used to explore the control of Rubisco in plants.

In addition, this work shows that MtnW and/or MtnU could be used as targets for any type of drug (including those derived from the lead above) destroying their activity. These enzymes are therefore excellent drug targets. Genomic comparisons show that these enzymes are present in the Bacillus cereus complex, including Bacillus anthracis. This can be seen for example using the SubtiList database and the Smith and Waterman alignments provided with the entries ykrW and ykrU and/or using the program Blast on the genomes displayed at the site GOLD (http://wit.integratedgenomics.com/GOLD/prokaryagenomes.html). One also observes many other pathogens in the case of MtnU (see below).

Analysis of gene expression demonstrates that expression of mtnU is always at least an order of magnitude smaller than that of mtnW, showing that the level of MtnW and MtnU are not related in a stoichiometric fashion.

Protein related to MtnU are ubiquitous and can be easily characterized. In particular motifs highly similar to the sequence ICYDIRFPE (with conservation of CY and an acidic residue) are the hallmark of proteins with related functions. These proteins are found in all three kingdoms of life, bacteria, Archaea and Eukarya (including Homo sapiens).

As a consequence this family of protein is a major drug target: modulation of its activity in different tissues of organisms will drastically alter their properties.

Knowledge of the pathway described herein in the agro-food domain permits improvement in a directed way of the growth yield of these bacteria and of any other organism possessing this pathway. In contrast, the same pathway may be intereferred with in pathogenic bacteria or parasites, or in unwanted plants and control their growth yield to a low level, eventually leading to their ultimate death. It will thus help fight diseases caused by relevant bacteria or parasites. In the medical domain, the knowledge of this pathway permits identification of several enzymes as potential targets for therapeutic drugs. In addition this identification permits the creation of diagnostic tests to identify bacteria having this pathway; including tests using DNA or protein arrays.

A noteworthy feature of the invention is that it uses the concept of neighborhood to explore hypotheses about gene functions. This concept permits one to construct links between apparently unrelated facts. The inventive activity results from putting together facts into a self-consistent picture not self-evident using present day knowledge. In the present invention, this strategy was used to identify at the gene and protein level, families of proteins which are involved in sulfur recycling. An important aspect of the invention, with the discovery of this pathway, is that it demonstrated that cells are easily limited in sulfur containing compounds. As a consequence, shutting off simultaneously several pathways for de novo sulfur molecules synthesis and/or recycling inhibits growth (cytostatic effect) and may lead to death. One important discovery associated with the invention is the demonstration that recycling (and scavenging compounds corresponding to the recycled metabolites) plays an essential backup role in the cell. This explains why these pathways have not been discovered previously: either one must know their existence beforehand, or one must interrupt the pathways for sulfur supply at several different steps at the same time to discover their existence and relevance. A special feature of the invention is that control of cell multiplication is therefore preferably obtained by interrupting at least two pathways simultaneously.

A special feature of this “double (resp. multiple)-bind strategy” is to create a new targeted approach to control proliferation of cells, microbial cells in particular. It can also be used in the control of pest plants in crop fields. In this case, this correspond to a strategic attack against a cell by combining two (or more) modes of inhibition. The invention illustrates this fact by the combination of the attack against methionine metabolism (for example using inhibitors of the one-carbon metabolism cycle, and/or inhibitors of methionine amino-peptidase, MAP), and the attack against MTA recycling. The originality of this part of the invention is that a simple attack on a pathway is generally insufficient, in particular in the case of interruption of different metabolic pathways starting from the same initial substrate and ending with the same product (pseudo-redundant pathways).

The present invention also embraces also the nucleotide sequences characterized in that they carry the information for the expression of the pathway of recycling of MTR, of mutants of these sequence or of fragments of these, able to form an immune complex with antibodies directed respectively against themselves.

The present invention also embraces to any recombinant sequence comprising sequences as defined above, possibly associated to a promoter cable to control the transcription of the DNA sequence as well as possibly coding for sequences of transcription termination and/or signals for optimizing translation and/or secretion.

The present invention also embraces the recombined nucleotide sequences, associated with a promoter and an operator permitting control of transcription and to a sequence signal permitting secretion of the corresponding proteins in the periplasmic space (in Gram negative bacteria) or in the external medium.

According to another aspect of the invention, the nucleotide sequences of the invention are able to hybridize with probes designed after nucleotide sequences of other chemicals polymers designed to hybridize to DNA, such as PNA (peptide nucliec acids) chains, having the nucleotide sequence indicated above. They may be used for diagnostic purposes.

The invention permits identification of molecules interfering with sulfur metabolism. The proteins according to the invention may be modelled using computers proteins and may be used as models for analyzing the interaction (“docking” in particular) with any type of molecule allowing modulation or inhibition of its the activity, of reference, the proteins expressed using any type of cloning, or in their natural context, may be studied for the inhibition of their activity. In particular, the methods of combinatorial chemistry and of phage display may be utilized for analyzing their inhibition. A preferred means for the analysis of the effect of putative inhibitors, is the study in vivo, in the bacterium B. subtilis, or ins a system reconstructed in vivo in an other organism (such as E. coli or yeast), is to study growth in the presence of MTR as sole sulfur source. The absence of growth indicates a inhibition. A favorable complementary means is to use a toxic analog of MTR (such as FMTR) in the presence of a poor sulfur source such as taurine, or in limiting sulfur growth conditions, and looking for the survival conditions of bacteria (or receptor organisms): any inhibition of the recycling pathway will be favorable to survival, which will provide a selective technique, for the identification of molecules of potential therapeutic interest.

Nucleotide sequences according to the invention are preferably obtained following usual cloning processes. Using PCR allows extension of the invention to cloning cognate genes from organisms sufficiently similar to B. subtilis. Alternatively the cloning is preferably performed in a B. subtilis strain disrupted for the appropriate genes of the pathway. The recombinant expression vectors for cloning able to transform an appropriate host cell also belong to the invention. These vectors comprise at least a part of a nucleotide sequence of the invention under the control of elements of regulation allowing its expression. Transformed microorganism strains are also within the scope of the invention. These strains host nucleotide sequences as defined above or recombinant vector(s) such as those defined above.

The proteins of the invention and their fragments, which may also be obtained by chemical synthesis, preferably present a high degree of purity and are used to form, according to well-known techniques, polyclonal and monoclonal antibodies.

Such polyclonal antibodies as well as monoclonal antibodies able to recognize specifically the proteins of the invention as well as their fragments are also part of the invention.

The invention also embraces the biological applications of nucleotide sequences, of the corresponding proteins as well as their fragments, and of the monoclonal or polyclonal. antibodies in particular for the construction of kits of diagnostic which could be constructed to identify organisms possessing all or part of the pathway for MTA recycling as described in the invention. These applications contain the elaboration, using intragenic fragments of the sequence (possibly discontinuous and containing ambiguities an/or analogs of standard nucleotides), of probes for the detection of similar sequences in the genes of organisms present in the pathway, whatever the organism, eubacteria, archebacteria or eucaryotes. This elaboration contains, notably, the denaturation of double strand sequences to obtain a monostrand sequence which can be used as a probe.

Appropriate probes for this type of detection are preferably labelled with a radio-active isotope (hot probes) or any other non radio-active group or reagent (cold probes) allowing the detection of the probe of interest hybridized with the preparation containing the DNA of interest. Among the radioactive probes used those which contain iodinated cytosine (with radioactive iodine) may be favored in the case when ultrasensitive methods for the detection of gamma photons would be available.

The invention also provides tools allowing fast detection, with high specificity, of similar sequences in genes coding for the enzymes of the MTA recycling pathway. These methods contain in vivo complementation studies, as described as well as hybridization and immunodetection.

For carrying out the detection methods considered above, based on the utilization of nucleotide probes, one preferably resorts to kits with the following:

a known quantity of a host nucleotide probe according to the invention,

preferably, a medium appropriate for, respectively, the formation of an hybridization reaction between the sequence to be identified and the probe,

preferably, reagents allowing the detection of hybridization complexes formed between the nucleotide sequence and the probe during the hybridization reaction.

The invention also embraces the immunological applications of the proteins defined above, in particular for the elaboration of specific antisera as well as polyclonal and monoclonal antibodies. The polyclonal antibodies are made according to the well-known techniques by injection of the protein into animals, recovery of the antisera for example using affinity chromatography. Alternatively the antibodies may be obtained by DNA vectors containing all or part of the genes of the invention and injection in animals.

The monoclonal antibodies are produced using techniques well-known in the art by fusing myeloma cells with spleen cells from animals previously immunized with proteins or derivatives of proteins of the invention.

All or part of the immunoprotective sequences of these proteins are preferably used for the elaboration of vaccines taking care not to give rise to unwanted immune reactions.

The present invention also provides a process of identifying compounds for activity against a bacilli infection by using at least one of the wild type genes of the bacilli as a target and a corresponding mutated gene or a recombinant bacteria carrying the wild type gene and a compound which may inhibit the activity of the genes.

### DESCRIPTION OF THE FIGURES

FIG. 1.

Location of transposon (Tn10) insertions in the mtn region. One insertion was localized 73 by upstream of the translational start point of the mtnK gene [6], four were located into mtnW and six into the mtnY gene. The insertion situated 353 bp downstream of the mtnW translation start point (strain BSHP7064) anal one situated 556 by downstream of the mtnY translation start point (strain BSHP7065) are shown in the figure.

FIG. 2.

Identification of the mtn region promoters by primer extension.

A. Identification of the transcription start site of the mtnKS operon. The size of the extended product is compared to a DNA-sequencing ladder of the mtnKS promoter region. Primer extension and sequencing reaction were performed with the same primer. The +1 site is marked by an arrow.

B. Identification of the transcription start site of the mtnU gene. The size of the extended product is compared to a DNA-sequencing ladder of the mtnU promoter region. Primer extension and sequencing reaction were performed with the same primer. The +1 site is marked by an arrow.

C. Identification of the transcription start site of the mtnV gene. The size of the extended product is compared to a DNA-sequencing ladder of the mtnV promoter region. Primer extension and sequencing reaction were performed with the same primer. The +1 site is marked by an arrow.

D. Identification of the transcription start site of the mtnWXYZ operon. The size of the extended product is compared to a DNA-sequencing ladder of the mtnWXYZ promoter region. Primer extension and sequencing reaction were performed with the same primer. Two +1 sites are marked by arrows.

FIG. 3.

Northern blot analysis of B. subtilis 168 mtnVWXYZ region. A total of 3 μg of RNA was used.

A. Northern hybridization with mtnV gene specific probe. RNA corresponding to lane 1 was obtained from a culture grown in minimal medium with sulfate as a sulfur source, and for lane 2 from a culture grown in minimal medium with methionine as a sulfur source.

B. Northern hybrydxzation with mtnW gene specific probe. RNA corresponding to lane 1 was obtained from a culture grown in minimal medium with sulfate as a sulfur source, and for lane 2 from a culture grown in minimal medium with methionine as a sulfur source.

C. Northern hybrpdixation with mtnZ gene specific probe. RNA corresponding to lane 1 was obtained from a culture grown in minimal medium with sulfate as a sulfur source, and for lane 2 from a culture grown in minimal medium with nxethionine as a sulfur source.

FIG. 4.

The MTR recycling pathway in B. subtilis.

FIG. 5.

Growth of mutants from the mtn region with MTR as sole sulfur source.

Panel A: ED1 minimal mediums plate with 1 mM IPTG containing 0.2 mM MTR as sole sulfur source WT, metI (BSIP 1143), mtnS (BSHP7010), mtnK (BFS1850), mtnU (BFS1851), mtnV (BSHP7020), mtnW (BSHP7014), mtnX (BFS1852), mtnY (BSHP7016) and mtnZ (BFS 1853) were inoculated for over-night growth at 37° C. No growth of mtnS, mtnK, mtnW and mtnY is represented by an example of absence of growth around a disc with MTR of mtnY mutant in panel B. Normal growth of mtnY and mtnX is represented by an example of normal growth around a disc with MTR of mtnV mutant in panel C. The partial growth of the mtnZ mutant is illustrated by its growth around a disc with MTR in panel C.

Panel B: The mtnY strain (BSHP7016) was inoculated on ED1 minimal medium plate with no added sulfur source. 10 μl of methionine (met) or MTR was put on paper discs anal the plate was incubated over-night at 37° C.

Panel C: The mtnY strain (BSHP7020) was inoculated on ED1 minimal medium plate with no added sulfur source. 10 μl of methionine (met) or MTR was put on paper discs and the plate was incubated over-night at 3?° C.

Panel D: The mtnZ strain (BFS1853) was inoculated on ED1 minimal medium plate with no added sulfur source. 10 μl of methionine (met) or MTR was adsorbed on paper discs and the plate was incubated over-night at 37° C. Methionine was used as a control.

FIG. 6.

Alignment of MtnX with the consensus of pfam00702 (http://www.ncbi.nlm.nih.gov/Structure/cdd/cddsrv.cgi?uid=pfam00702&version=v1.54), that includes L-2-haloacid dehalogenase, epoxide hydrolases and phosphatases. Red letters represent identities, blue letters conservative replacements (similarity classes: AGPST, ILMV, FWY, DENQ, HKR,). A loop containing a metal (presumably iron, or an iron-sulfur cluster) is likely to be present in MtnX.

FIG. 7.

Toxicity of MTR for BSHP7014 strain. Strain BSHP7014 (mtnW:lacZ amyE::pxyl mtnXYZ) was grown on ED1 minimal medium plates in the presence of sulfate as sulfur source (panel A) or in the absence of any added sulfur source (agar as sole sulfur source, panel B). Xylose was added to the medium in order to trigger the expression of mtnXYZ from the pxyl promoter, 10 μl of methionine (met) or MTR was adsorbed on paper discs and plates were incubated over-night at 37° C. Methionine was used as a control for growth and/or toxicity of the sulfur source.

FIG. 8.

Alignment of MtnZ with the consensus of pfam03079 (http://www.ncbi.nlm.nih.gov/Structure/cdd/qrpsb.cgi?RID=1014604213-20481-4181), coding for aci-reductone enzymes. Red letters represent identities, blue I letters conservative replacements (classes: AGPST, ILMV, FWY, DENQ, HKR).

### DETAILED DESCRIPTION OF THE INVENTION

Transposon Insertion Mutations and Phenotype of Inactivated Mutants

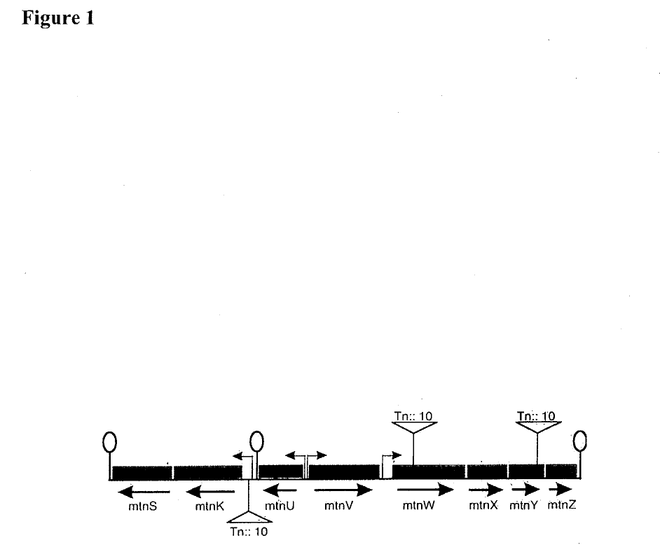

Mutants were obtained by transformation of a wild type strain with a random transposon library, selecting for growth in the presence of trifluoromethylthioribose (3F-MTR). The mutants were subsequently tested for growth on plates lacking sulfur source but supplemented with MTR: only those that could not grow were retained for further study. In order to ascertain that the resistant phenotype was not coming from secondary mutations but was directly related to the transposon insert, the chromosome DNA was extracted from each putative mutant and back transformed into a wild type strain selecting for the transposon antibiotic marker. The 3F-MTR and MTR phenotypes were subsequently tested and only those mutants that passed the test were retained. The insertion positions of the transposons were then sequenced. As shown in FIG. 1 we recovered mutants in several genes located in the close vicinity of each other. One mutant was located at the mtnK locus (previously named ykrT [6]), four were located into ykrW and six into the ykrY gene. One clone with transposon insertion into the ykrW gene (strain BSHP7064, insertion situated 353 bp downstream of ykrW translation start point) and one into the ykrY gene (strain BSHP7065, insertion situated 556 bp downstream of ykrY translation start point) were retained for further studies.

Using the collection of mutants constructed during the Bacillus subtilis functional analysis, program (http://locus.jouy.inra.fr/cgi-bin/genmic/madbase/progs/madbase.oper] and http://bacillus.genome.adjp, [9]) and constructing mutants which were not available in the collection, we tested all genes in the region for their phenotype of growth on MTR as the sole sulfur source. Table 1 displays the results obtained. As we can see, mutants in mtnK (previously identified as coding for MTR kinase [6], strain BFS1850), mtnS (strain BSHP7010 [6]), ykrU (renamed mtnU, strain BFS1851), ykrW (renamed mtnW, strain BSHP7014 allowing the expression of downstream genes) and ykrY (renamed mtnY, strain BSHP7016) failed to grow on the substrate. In the absence of IPTG ykrX (renamed mtnX, strain BFS1852) also failed to grow, but it recovered its growth properties when IPTG was added to the medium, suggesting some polar effect of the transposon insertion. The mutant of ykrZ (renamed mtnZ, strain BF51853) presented only a very weak (residual) growth on MTR, suggesting the presence in the cell of some other enzymatic activity able to partially complement the lack of mtnZ gene product. Disruption of ykrV (renamed mtnV, strain BSHP7020) had no visible effect on growth on MTR as the sulfur source.

Identification of Promoters

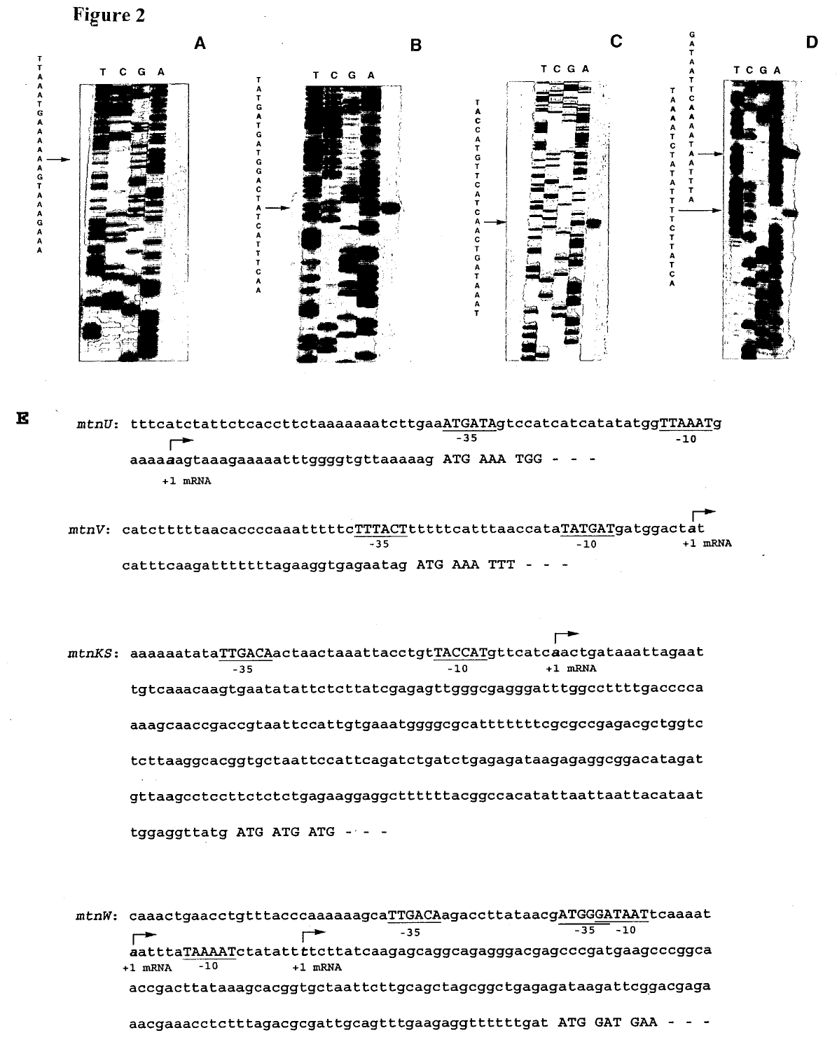

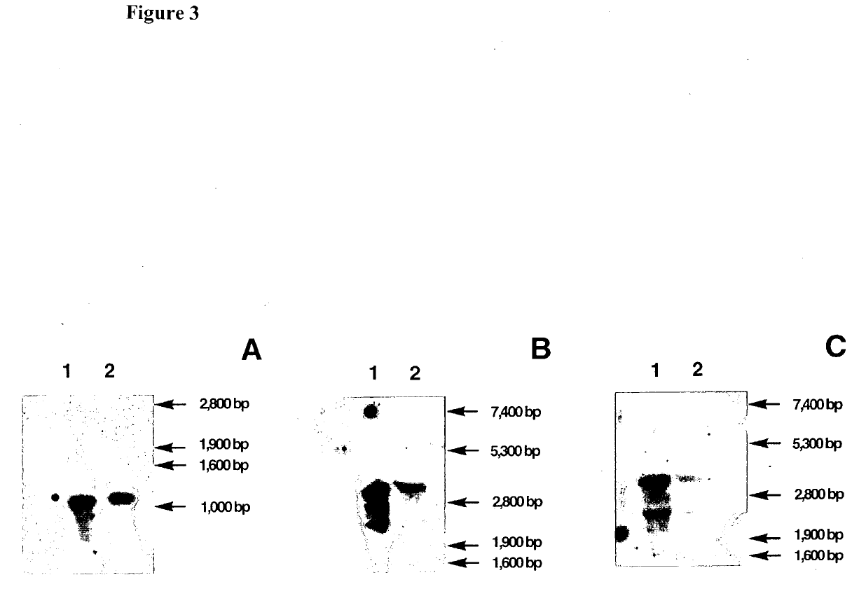

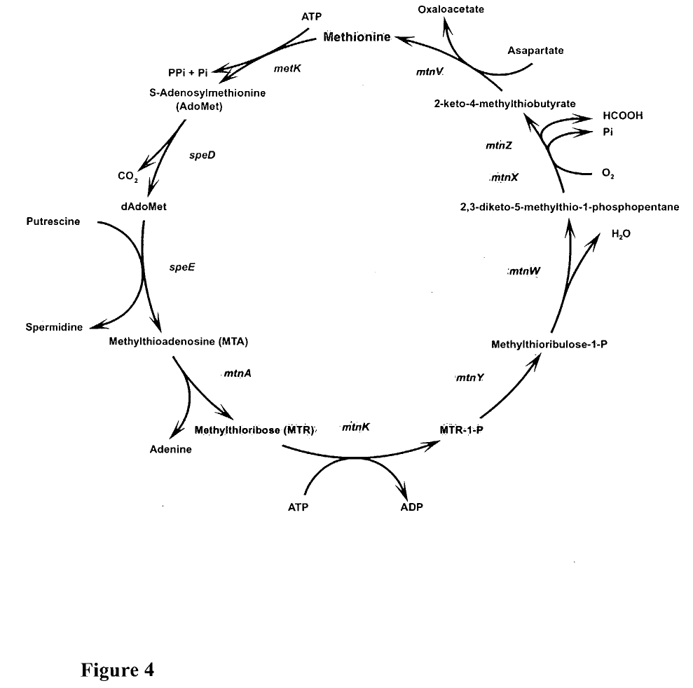

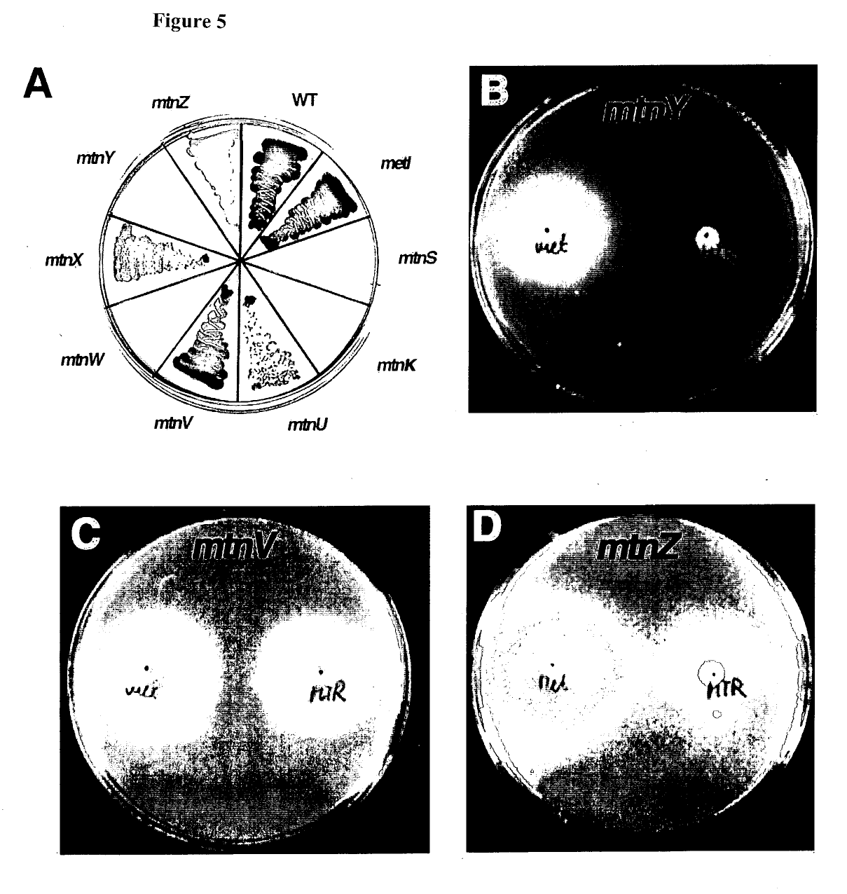

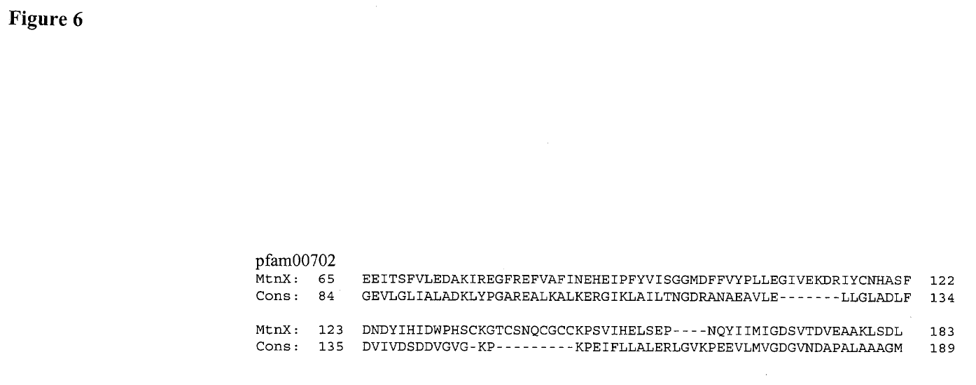

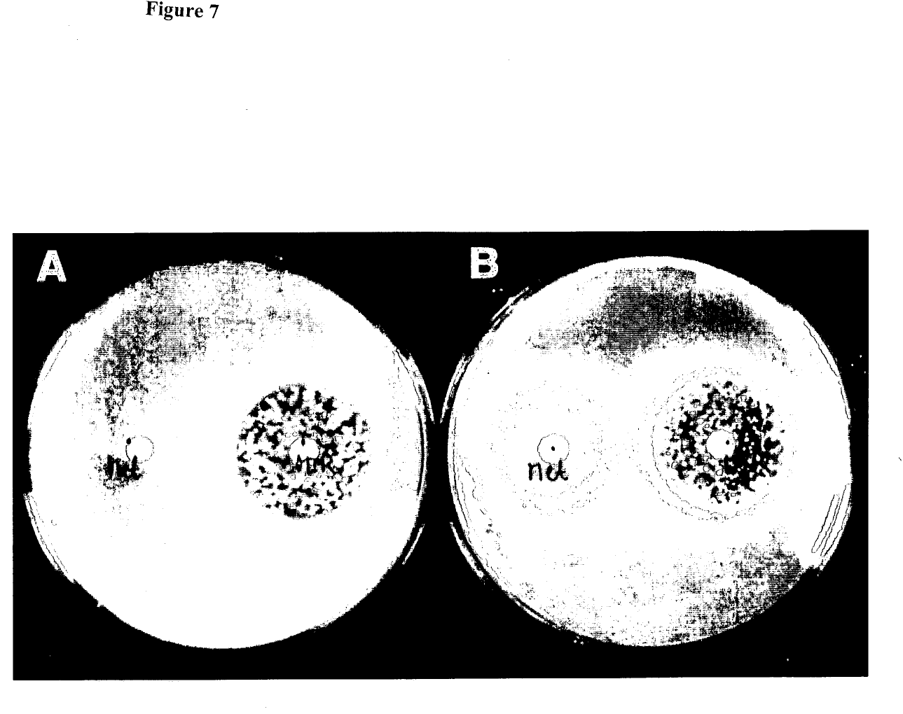

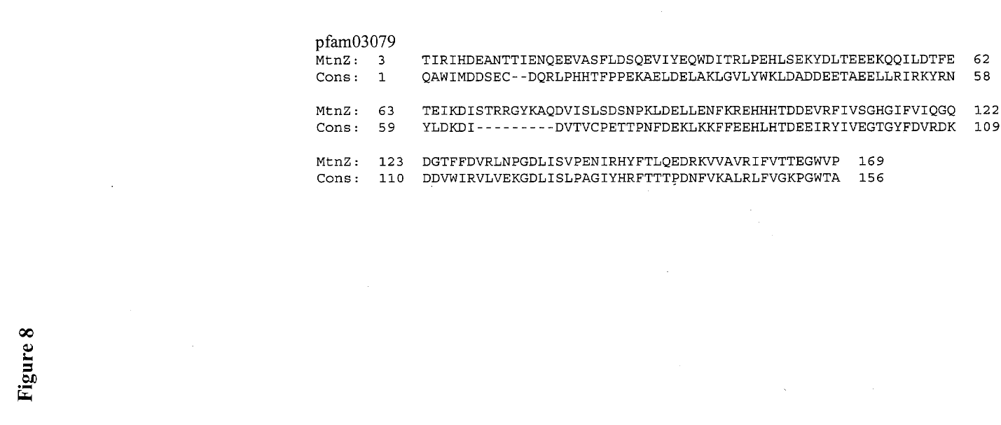

Several genes in the region have been shown by Henkin and co-workers to be expressed from promoters regulated by the S-box attenuation system [7]. This is the case of mtnKS and mtnWXYZ transcription units. Some of the genes, however, are not regulated in this way. Expression of the mtnU and mtnV genes is not subject to that regulation since no S-box is present in their leader transcript. As shown in FIG. 2A the promoter of mtnU is located 35 nt from the translation start point. Its start was found to lie 5 nt downstream from a putative −10 box identified in the sequence (TTAAAT). Upstream from this box separated by 18 nt is a −35 box (ATGATA) with sequence similar to the consensus sequence TTGACA that is typical of B. subtilis sigma A-dependent promoters [10].

The promoter of mtnV is located 42 nt upstream from the translation start point. Its start lies 8 at downstream from a putative −10 box identified in the sequence (TATGAT) separated by 17 nt from −35 box (TTTACT) (see FIG. 2.B). The mtnU and mtnV genes share the same promoter region (94 nt) but are transcribed in divergent orientation from overlapping promoters. Thus, the −10 box of the mtnV promoter is situated between the −10 and −35 boxes of the mtnU promoter and the −10 box of the mtnU promoter is situated between the −10 and −35 boxes of the mtnV promoter.

The mtnKS promoter region is 326 nt long. Its start was found to lie 7 nt downstream from a putative −10 box identified in the sequence (TACCAT) (see FIG. 2.C). Upstream from this box and separated by 18 nt is a −35 box (TTGACA), a typical B. subtilis sigma A-dependent promoter. Downstream of this promoter lies an S-box regulatory sequence.

Genes mntWXYZ are expressed from two overlapping promoters that are situated in a 195 nt long region. The upstream P1 promoter's start was found to lie 7 nt downstream from a putative −10 box identified in the sequence (GATAAT) separated by 17 nt from a consensus −35 box (TTGACA). The promoter's start was found to lie 7 nt downstream from a putative −10 box identified in the sequence (TAAAAT) upstream from which is a −35 box (ATGGGA) (see FIG. 2.D). The −10 box of the P1's promoter and the −35 box of P2 are partly overlapping. The relative intensity of the signals indicates that transcription from the P1 promoter is more abundant than from P2 (see FIG. 2.D).

Transcription Organization of the mtn Locus

To further investigate transcription of the mtnVWXYZ genes, RNA synthesis was analyzed by Northern blotting. RNA was extracted from exponentially growing cells, in minimal medium containing either sulfate or methionine as sulfur source. As shown in FIG. 3.A, a band of about 1200 nt, corresponding to the expected length of a transcript initiated at the mtnV promoter and terminating near its stop codon, was observed for the mtnV gene probe. An equal intensity of the signal was observed for mtnV transcripts prepared from cells either grown with sulfate or with methionine as sulfur source (lanes 1 and 2, FIG. 3.A).

When mtn W and mtnZ gene specific probes were used, two bands were revealed: one of about 2.5 kb and second of about 3.2 kb (FIGS. 3.B and 3.C). The larger band corresponds to the expected length of a transcript initiated at the mtnW promoter and terminating in a stem and loop structure at the end of the mtnWXYZ transcriptional unit. The smaller band can possibly be the result of RNA processing at the end of the S-box regulatory sequence of the 5′ extremity of the transcript. The intensity of bands when hybridizing RNA from cells grown with sulfate as sulfur source was higher than when using RNA from cells grown in the presence of methionine (lane 1 and 2 in FIGS. 3.B and 3.C).

As shown previously, mtnK and mtnS are expressed as an operon, while mtnU is expressed independently [6].

Regulatory Features

To substantiate the results obtained with RNA analysis and further investigate the expression of genes from the mtn region, we constructed mutants earring lacZ transcriptional fusions as well as used some mutants constructed during the functional analysis program (also corresponding to lacZ transcription fusions). Table 2 shows the results obtained with these strains when using sulfate or methionine as sulfur source. The mtnU gene (strain BFS1851, mtnU::lacZ) is expressed constitutively at a fairly low level and its expression is independent of the sulfur source used (62 U/mg of protein in the exponential growth phase in presence of sulfate and 53 U/mg of protein in the exponential growth phase in presence of methionine). In contrast, the mtnV gene (strain BSHP7020, mtnV::lacZ) although expressed in the similar way (constitutive and sulfur source independent expression) is expressed at a significantly higher level (217 U/mg of protein in the exponential growth phase in presence of sulfate and 181 U/mg of protein in the exponential growth phase in presence of methionine).

The genes from the mtnWXYZ transcriptional unit (strains BSHP7014, BFS1852, BSHP7016 and BFS1853 for mtn W::lacZ, mtnX::lacZ, mtnY::lacZ and mtnZ:lacZ, respectively) are expressed in a coordinated and sulfur source-dependent way. The expression of the first gene in the operon (mtnW) is higher than that of the last one (mtnZ) with intermediary values for intermediary genes mtnX and mtnY. This suggests the effect of some transcription attenuation during the process of transcription (see Table 2). A 5-fold difference is observed between the expression of the mtnWXYZ genes in the presence of sulfate and that in the presence of methionine (579 U/mg of protein in the exponential growth phase in the presence of sulfate and 113 U/mg of protein in the exponential growth phase in the presence of methionine for the mtnW::lacZ transcriptional fusion and 280 U/mg of protein in the exponential growth phase in presence of sulfate and 57 U/mg of protein in the exponential growth phase in presence of methionine for mtnZ::lacZ transcriptional fusion). This observation is in accordance with the presence of S-box regulatory element in the promoter region of mtnWXYQ operon which modulates gene expression as a function of methionine availability [7].

Reconstruction of the Metabolic Pathway

In order to identify the methionine salvage pathway we made constructs allowing us to decipher the order of the gene products in the pathway, together with in silico, physiologic and genetic analysis of the effect of metabolites of the pathway. This is reminiscent of the way advocated by Koonin et al, for the use of in silico approaches as complement to in vivo experiments [11].

As a first goal we showed that the end product of the pathway is indeed methionine. This was demonstrated by showing that MTR, which is a good sulfur source, can be used as the methionine source in methionine auxotrophs (FIG. 4, FIG. 5 and data non shown).

Two genes in the pathway are dispensable, mtnV and mtnX. The first one encodes a transaminase of which there are nine putative paralogs in the genome of B. subtilis (YwfG, AlaT, AspB, PatA, YhdR, YdfD, PatB, YisV, and HisC). In the same way, MtnX (YkrX) is a member of the phosphatase family pfam00702 ([12], FIG. 6), and therefore of a ubiquitous class of hydrolases (several phosphatase genes, in particular are present in the genome of B. subtilis). This is likely to account for the lack of phenotype under our growth conditions. Inactivation of mtnZ provides only a very weak, residual growth on MTR. Inactivation of mtnK, mtnS, mtnY and mtnW result in resistance to 3F-MTR and lack of growth on MTR. Inactivation of mtnW with a polar effect on the distal genes (by insertion of a disrupting plasmid) has a phenotype similar to that of mtnY (i.e. lack of growth on MTR, and lack of influence of MTR on sulfate supplemented plates). In contrast, we discovered that MTR is toxic when the distal genes are present (when used as sole sulfur source or in the presence of sulfate, see FIG. 7). Because of the weak phenotype of a mtnZ mutant and the absence of phenotype of a mtnX mutant, we can be confident that MtnY acts before MtnW (this is a common feature in operons, where it is generally observed that the more distal genes code for proteins acting in the more proximate steps of the pathway).

The methionine salvage pathway has been deciphered in K. pneumoniae. It is possible, combining this knowledge to the genetic and physiologic results just described, to use it at the basis for reconstructing in silico the corresponding metabolic pathway in B. subtilis. The first steps are similar in both organisms: methylthioadenosine is converted into MTR by a nucleosidase (MtnA, [5]). Subsequently, MTR is phosphorylated into MTR-1-phosphate by MtnK [6]. On the other end of the pathway, methionine is synthesized directly from its keto acid precursor, 2-keto-4-methylthiobutyrate, by a transaminase. MtnV is the likely preferred enzyme for this activity. In K. pneumoniae a dioxygenase is converting 2,3-diketo-b-methylthio-1-phosphopentane into 2-keto-4-methylthiobutyrate [2]. Using dynamic programming (FASTA) we compared the sequence of the corresponding protein to the complete proteome of B. subtilis. ykrZ comes out as the first hit, as the most similar enzyme present in the proteome. Furthermore, it displays a strong consensus similarity with the dioxygenases of the family pfam03079 (FIG. 8) [12]. In order to check whether dioxygen was indeed involved in the case of B. subtilis we grew the cells anaerobically, with nitrate as an electron acceptor, and tested for growth on MTR: while the wild type strain grew well when sulfate was the carbon source, it failed to grow with MTR (Table 1).

Since this dioxygenase is coded in the mtn operon we can infer that it indeed displays the corresponding activity [11], and we therefore renamed it MtnZ. In K. pneumoniae, the immediate precursor activity is that of a coupled phosphatase. The presence of MtnX, which belongs to family pfam00702 comprising phosphatases is strongly suggestive of its involvement at this step [12]. We are thus left with two enzymes, and two steps. We also know, from the genetic data, that the steps are catalyzed in the order MtnY, MtnW. Finally, the reaction, needed upstream of MtnZ is active on a molecule phosphorylated in position 1. MtnW is very similar to ribulosephosphate carboxylase oxygenase (Rubisco). It is therefore likely to be active on a ribulose-1-phosphate derivative. Hence MtnY, which is similar to the araD gene product of E. coli (ribulose-5-phosphate epimerase) is most likely to be an epimerase that converts MTR-1-P into 5-thiomethyl-ribulose-1-phosphate, which is the substrate of MtnW. This is strongly supported by the list of similarities found about this gene at the SubtiList database (http://genolist.pasteur.fr/Subtilist).

At this stage it is difficult to explicitly identify the activity of MtnW. Even in the case of the paradigmatic Rubisco, with many crystal structures known, the exact mechanism of catalysis is still a matter of controversy. However we can note (as did [8]) that all the residues involved in catalysis have been conserved, the only residues modified being those involved in the binding of the phosphate at position 5 of ribulose diphosphate. The reaction is that of a dehydratase, but the pathway of the reaction is not yet known. Further work will establish the details of the reaction.

Finally MtnU is also defective for MTR recycling. However, this protein is synthesized at a level much lower that that of the other components of the pathway. We can therefore surmise that it is involved in a regulatory step in the pathway.

Several genes in the vicinity of mtnK have been shown to have significant relationships with sulfur metabolism. In particular, it has been known for some time that genes ykrWXYZ were preceded by an S-box, typical of sulfur mediated regulation [7]. In addition, while analyzing the function of ribulose-1,5-diphosphate carboxylase (Rubisco), Hanson and Tabita discovered a class of highly related enzymes that were involved in sulfur metabolism [8].

The MTR analog trifluormethylthioribose is toxic if the methyl sulfur moiety of the molecule is recycled [13]. This molecule was therefore an excellent candidate to explore the steps needed for MTR recycling: resistant mutants were found in genes mtnK, mtnW and mtnY. Remarkably, no permease gene was found, suggesting that MTR enter the cells via several entries. In addition, apart from the mtnKS and mtnWXYZ operons no other genes was found, suggesting that all essential steps for recycling are coded for by these genes (or that other steps are coded for by redundant genes). The first step of the metabolic pathway is phosphorylation of MTR. The last step presumably, is transamination, with mtnV being the preferred transaminase.

Interestingly, the pathway described in this work, although similar to that found by the pioneering work of Abeles and co-workers, uses an original enzyme, MtnW, which is extremely similar to Rubisco [14, 15]. The corresponding activity is known to exist in K. pneumoniae, but no corresponding enzyme has yet been isolated. Furthermore, while most of the genome sequence of this bacterium is known (http://wit.integratedgenoniics.com/GOLD/) no counterpart of MtnW could be found (data not shown). As discovered by Hanson and Tabita, MtnW counterparts constitute a special class (class IV) of Rubisco-like enzymes, which are involved in sulfur metabolism: we can presume that they are all part of the methionine salvage pathway in these organisms [8]. Interestingly, the expected reaction required to metabolize 5-thiomethyl-ribulose-1-phosphate is that of a dehydratase that may use a co-factor as a substrate for the reaction [16]. Rubisco, in the presence of carbon dioxide (resp, dioxygen), acts as a carboxylase (resp, dioxygenase) which cleaves the substrate. In the present case we expect that, instead of cleavage, we have maintenance of a five carbon molecule that is dephosphorylated (by MtnX) and subsequently cleaved by dioxygen in the reaction mediated by MtnZ.

As a strong support of this schema, we found counterparts of MtnK and of MtnZ in K. pneumoniae, substantiating the proposed pathway. In this latter organism the counterpart of MtnY is not known, and the corresponding step (opening of the MTR-1-P ring with epimerization) is not known in any organism yet. MtnY is part of a very wide family of aldolases-epimerases-transketolases and in silico prediction of function alone, at this stage is highly problematic (wrong assignment is frequent for similar functions [17]), but combination with genetic data make the prediction highly probable [11]. We therefore propose that MtnY be used as a basis for annotation of similar gene products, For example in Xylella fastidiosa, gene XF2209 and in Pseudomonas aeruginosa gene PA1683, probably encodes the cognate activity. Noticeably, a counterpart exists in the Human Genome, where a similar pathway operates.

Two gene products are not directly accounted for in the present schema, MtnU and MtnS. MtnU is expressed at a very low level (ten times lower) as compared to MtnW, and this would hardly fit with the expected stoichiometric enzyme concentration usually found in multistep metabolic pathways. In addition, we found that its synthesis is not submitted to any regulation by the sulfur source. Similarly, MtnS, which is highly similar to an eukaryotic translation initiation factor eIF-2B involved in GTP/GDP exchange is a member typical of a class of GTP-dependent regulators. The presence of two regulator molecules in this pathway indicates that it must have an important role in the cell. B. subtilis is likely to strive on the phylloplane. It is therefore regularly submitted to very high local concentrations of oxygen, and we speculate that this pathway, in addition to providing an excellent means to recycle the energy costly methionine, is used as a means to protect the cell against oxygen.

In conclusion, this work demonstrates that a complete methionine salvage pathway exists in B. subtilis. This pathway is chemically similar to that in K. pneumoniae, but recruited different proteins to this purpose. In particular a paralogue or Rubisco, MtnW, is used at one of the steps in the pathway. A major observation stemming from the present experiments is that in the absence of MtnW MTR becomes extremely toxic to the cell. This sensitivity opens an unexpected target for never antimicrobial drugs, since analogs of 5-methylthio-ribulose1-phosphate might have a strong inhibitory effect on growth on bacteria containing this methionine salvage pathway, including Bacillus anthracis.

Materials and Methods

Bacterial strains and plasmids, and growth media: E. coli and B. subtilis strains as well as plasmids used in this work are listed in Table 3. E. coli TG1 and XL1-Blue were used for cloning experiments (TG1 for single cross-over recombination and XL1-Blue for double cross-over recombination). Despite the fact that there are no public regulations yet in this domain in China, all experiments were performed in accordance with the European regulation requirements concerning the contained use of Genetically Modified Organisms of Group-I (French agreement N^{o} 2735). E. coli and B. subtilis were grown in Luria-Bertani (LB) medium [18] and in ED minimal medium: K₂HPO₄, 8 mM; KH₂PO₄, 4,4 mM; glucose, 27 mM; Na₃-citrate, 0.3 mM; L-glutamine, 15 mM; L-tryptophan, 0.244 mM; ferric citrate, 33.5 μM; MgSO₄, 2 mM; MgCl₂, 0.61 mM; CaCl₂, 49.5 μM; FeCl₃, 49.9 μM; MnCl₂, 5.05 μM; ZnCl₂, 12.4 μM; CuCl₂, 2.52 μM; CoCl₂, 2.5 μM; Na₂MoO₄, 2.48 μM. When methionine was used as sulfur source (1 mM), MgSO₄ was replaced by MgCl₂ at the same magnesium concentration (2 mM). For assaying growth on plates, either the MgSO₄ containing medium or the sulfur-free basal medium was used (MgSO₄ was replaced by MgCl₂ as described above). In the latter case, 10 [μl of the sulfur source under investigation was applicated onto paper discs (MTR, 200 mM stock solution and methionine, 100 mM stock solution) deposited at the center of the plate, after bacteria had been uniformly spread at the surface of the plate, and growth was measured around the disK. In some cases MTR was used directly in the plate as sulfur source (0.2 mM). When necessary IPTG was included at 1 mM concentration. When xylose was added to the medium (0.5%) in order to trigger the expression of genes under the control of Pxyl inducible promoter, fructose was used as carbon source instead of glucose. LB and ED plates were prepared by addition of 17 g/liter Bacto agar or Agar Noble (Difco), respectively, to the medium. When included, antibiotics were added to the following concentrations: ampicillin, 100 mg/liter; chloramphenicol, 50 mg/liter; spectinomycin, 100 mg/liter; erythromycin plus lincomycin, 1 mg/liter and 25 mg/liter. Bacteria were grown at 37° C. The optical density (OD) of bacterial cultures was measured at 600 nm. MTR was prepared from MTA (Sigma, D5011) by acid hydrolysis as described by Schlenk [19]. 3-fluoromethythiorybose (3F-MM, 5-thio-5-5-S-trifluoromethyl-D-ribose) was synthesised accordingly to [6, 20]. When added directly to the ED1 medium plate, 3F-MTR was used at 100 mg/liter concentration and when applicated onto paper discs 100 mM stock solution was used. For anaerobic growth on plates, the Anaerocult A (Merck) within an anaerobiosis jar for CO₂ production with simultanious O₂ absorbtion was used. Sulfur-free ED1 minimal medium plates were supplemented to 1% glucose final concentration and with 0.5% sodium pyruvate and 20 mM sodium nitrate as electron acceptor. Plates were incubated at 37° C. for 4 days with the sulfur source under investigation.

Transformation: Standard procedures were used to transform E. coli [21] and transformants were selected on LB plates containing ampicillin, spectinomycin or ampicillin plus spectinomycin. B. subtilis cells were transformed with plasmid DNA following the two-step protocol described previously [22]. Transformants were selected on LB plates containing erythromycin plus lincomycin or spectinomycin or chloramphenicol.

Molecular genetics procedures: Plasmid DNA was prepared from E. coli by standard procedures [21]. B. subtilis chromosomal DNA was purified as described by Saunders [23]. Restriction enzymes and T4 DNA ligase were used as specified by manufacturers.

DNA fragments used for cloning experiments were prepared by PCR using PfuTurbo DNA polymerase (Stratagene). Amplified fragments were purified by QIAquick PCR Purification Kit (Qiagen). DNA fragments were purified from a gel using Spin-X columns from Corning Costar by subsequent centrifugation and precipitation.

The ykrXYZ region (nucleotides -31 relative to the ykrX translation start point and ending 3 by after the stop codon of ykrZ) was amplified by PCR using primers introducing a SpeI cloning site at the 5′ end and a BamHI cloning site at the 3′ end of the fragment. This fragment was then inserted into the SpeI and BamHI sites of xylose-inductible pX plasmid [24] producing plasmid pHPP7015. Prior to transformation, this plasmid was linearised at its unique ScaI site. Complete integration of the plasmid was obtained by a double cross-over event at the amyE locus, giving strain BSHP7015.

The DNA downstream from the ykrW gene (nucleotides +41 to +257 relative to the translation start point) was amplified by PCR using primers introducing an EcoRI cloning site at the 5′ end and a BamHI cloning site at the 3′ end of the fragment, then inserted into the EcoRI and BamHI sites of plasmid pJM783 [25] producing plasmid pHPP7014. To introduce an additional antibiotic resistance gene into plasmid pHPP7014, a SmaI restricted spectinomycin resistance cassette [26] was inserted into the ScaI restriction site of the bloc gene producing plasmid pHPP7014bis. The plasmid in which the mtnW gene was disrupted as well as fused (transcriptional fusion) with the lacZ gene was introduced into the chromosome of BSHP7015 strain by a single cross-over event, giving strain BSHP7014.

For transcriptional fusion of mtnY with the lacZ gene, a DNA segment downstream from the mtnY gene (nucleotides +57 to +264 relative to the translation start point) was amplified by PCR using primers introducing an EcoRI cloning site at the 5′ end and a BamHI cloning site at the 3′ end of the fragment, then inserted into the EcoRI and BamHI sites of plasmid pJM783 producing plasmid pHPP7016. The plasmid in which the mtnY gene was disrupted as well as fused (transcriptional fusion) with the lacZ gene was introduced into the chromosome by a single cross-over event, giving strain BSHP7016.

To construct a mtn V transcriptional fusion with the lacZ gene, a DNA fragment downstream from the mtnV gene (nucleotides +44 to +259 relative to the translation start point) was amplified by PCA using primers introducing an EcoRI cloning site at the 5′ end and a BamHI cloning site at the 3′ end of the fragment, then inserted into the EcoHI and BamHI sites of plasmid pJM783 producing plasmid pHPP7011. The plasmid in which the mtnV gene was disrupted as well as fused (transcriptional fusion) with the lacZ gene was introduced into the chromosome by a single cross-over event, giving strain BSHP7020.

Within the framework of a European Union and Japanese projects for the functional analysis of the genome of B. subtilis, more than 2000 genes have been disrupted by fusion with the lacZ reporter gene (http://locus.jouy.inra.fr/cgi-bin/genmic/madbase/progs/madbase.oper1 and http ://bacillus.genome.ad.jp). The strains from the collection used in this study, constructed by Dr S. Krogh, are listed in Table 3.

Transposon mutagenesis: A transposon bank was constructed by introduction of the mini-Tn10 delivery vector pIC333 (27) into the B. subtilis 168 strain as described previously [28]. Several thousand independent clones were pooled together and 5 Samples of chromosomal DNA were prepared for further use. To obtain 3F-MTR resistant clones, B. subtilis 168 was transformed with chromosomal DNA containing previously prepared transposon banks and clones were selected on LB plates containing spectinomycin. Then, using velvets replicas, clones were transferred onto minimal medium plates containing 3F-MTR at 100 μM concentration and allowed to grow for 24 hrs. The single transposon insertion event was confirmed by back-cross into strain 168 and check for 3F-MTR resistance. To determine the location of the transposon insertion, chromosomal DNA was prepared, followed by subsequent digestion with HindIII, self ligation in E. coli XL1-Blue strain and plasmid sequencing. The primers used for sequencing of transposon insertions were the followings: Tn10 left: 5′GGCCGATTCATTAATGCAGGG3′ and Tn10 right: 5′CGATATTCACGGTTTACCCAC3′.

RNA isolation and manipulation: Total RNA was obtained from cells growing on ED1 minimal medium with sulfate or methionine as sulfur source to an OD₆₀₀ of 0.5 using “High Pure RNA Isolation Kit” from Roche. The RNA concentration was determined by light absorption at 260 nm and 280 nm. 2 μg of RNA, were loaded onto 1.2% agarose gel to check the RNA purity and integrity.

RNA molecules were separated on 1% agarose gels and transfered to nylon membranes (Hybond-N, Amersham). Efficiency of transfer was monitored by analysis of ethidium bromide-stained material. Membranes were prehybridized at 50° C. for 1 hr in DIG Easy Hyb buffer from Roche. Hybridization was performed under the same conditions with mtnV, mtnW or mtnZ specific probes using a non-radioactive DNA labeling and detection kit “Dig-UTP labeling” from Roche.

Primer extension analysis using reverse transcriptase AMV (Roche) was performed as described by [29] with two oligonucleotides for each promoter identification. For mtnKS promoter the followings primers were used:

5′ACCAGCGTCTCGGCGCGAAAAAAATGCGCCCC3′ and 5′TCACAATGGAATTACGGTCGGTTGCTTTTGG3′ (+137 to +169 and +172 to +203 with respect to the translation start point, respectively; for the mtnU promoter the following primers were used: 5′AGTTCATCAAGATTGGCCAGATCATATCCG3′ and 5′CAGGCAGAACAAGAACATCAGCATGTTTGC′ (−133 to −103 and −90 to −60 with respect to translation start point, respectively); for the mtnV promoter the followings primers were used: 5′GTTTCATCTCCTCAACAATATGCTCAGGAG′ and 5′TCCCAGATTGATAACGTCATGTCCTTCTGC′ (−166 to −146 and −114 to −84 with respect to the translation start point, respectively); for the mtnWXYZ promoter the followings primers were used: 5′CGTTTCTCGTCCGAATCTTATCTCTCAGCC′ and 5′AGCTGCAAGAATTAGCACCGTGCTTTATAAG′ (+43 to +73 sad +76 to +107 with respect to the translation start point, respectively). The same primers were used for the generation of sequence ladders. Reaction products were separated on 7% denaturing polyacxylamide gel containing 8 M urea. DNA sequences were determined using Sanger's dideoxy chain-termination method with “Thermo Sequenase radiolabeled terminator cycle sequencing kit” from Amersham Pharmacia Biotech.

Enzyme assays: B. subtilis cells containing lacZ fusions were assayed for β-galactosidase activity as described previously [30]. Specific activity was expressed in Units per mg protein. The Unit used is equivalent to 0.28 nmols min⁻¹ at 28° C. Protein concentration was determined by Bradford's method using a protein assay Kit (Bio-Rad Laboratories). At least two independent cultures were monitored.

Amylase activity was detected after growth of B. subtilis strains on Tryptose Blood Agar Base (TBAB, Difco) supplemented with 10 g/liter hydrolyzed starch (Sigma). Starch degradation was detected by sublimating iodine onto the plates.

### Deposit of Biological Materials

The following materials have been deposited at the CNCM (Collection Nationale De Cultures De Micro-organisms, Institut Pasteur, 28, rue du Dr Roux, 75724 Paris Cedex 15, France):

BSHP 7016 mtnY (ykrY) CNCM I-2858 genotype trpC2 mtnY::lacZ

BSHP 7014 mtnW (ykrW) CNCM I-2859 genotype trpC2 mtnW::lacZ

BFS 1851 mtnU (yrkU) CNCM I-2860 genotype trpC2 mtnU::lacZ

The deposits are incorporated herein by reference.

Abbreviations used herein: bp: base pairs; CDS: coding sequence; IPTG: isopropyl β-D-thiogalactopyranoside; kb: kilobase; MTA: methylthioadenosine; MTR: methylthioribose; 3F-MTR: trifluoromethylthioribose; nt: nucleotides.

### REFERENCES

1. H R Schroeder, C J Barnes, R C Bohinski, M F Mallette: Biological production of 5-methylthioribose. Can J Microbiol 1973, 19:1347-1354.

2. Y Dai, T C Pochapsky, R H Abeles: Mechanistic studies of two dioxygenases in the methionine salvage pathway of Klebsiella pneumoniae. Biochemistry 2001, 40:6379-6387.

3. J Heilbronn, J Wilson, B J Berger: Tyrosine aminotransferase catalyzes the final step of methionine recycling in Klebsiella pneumoniae. J Bacteriol 1999, 181:1739-7.'147.

4. M K Riscoe, A J Ferro, J H Fitchen: Analogs of 5-methylthioribose, a novel class of antiprotozoal agents. Antimicrob Agents Chemother 1988, 32:1904-1906.

5. A Sekowska, A Danchin: Identification of yrrU as the methylthioadenosine nucleosidase gene in Bacillus subtilis. DNA Res 1999, 6:255-264.

6. A Sekowska, L Mulard, S Krogh, J K Tse, A Danchin: MtnK, methylthioribose kinase, is a starvation-induced protein in Bacillus subtilis. BMC Microbiol 2001, 1:15.

7. F J Grundy, T M Henkin: The S box regulon: a new global transcription termination control system for methionine and cysteine biosynthesis genes in gram-positive bacteria. Mol Microbiol 1998, 30:737-749.

8. T E Hanson, F R Tabita: A ribulose-1,5-bisphosphate carboxylase/oxygenase (RubisCO)-like protein from Chlorobium tepidum that is involved with sulfur metabolism and the response to oxidative stress. Proc Natl Acad Sci USA 2001, 98:4397-4402.

9. V Vagner, E Dervyn, S D Ehrlich: A vector for systematic gene inactivation in Bacillus subtilis. Microbiology 1998, 144:3097-3104.

10. C P Moran: RNA polymerase and transcription factors. In: Bacillus subtilis and Others Gram-Positive Bacteria Edited by A L Sonenshein. Washington, D.C.; American Society for Microbiology; 1993: 653-667.

11. L M Iyer, L Aravind, P Bork, K Hofmann, A R Mushegian, I B Zhulin, E V Koonin:. Quad erat demonstrandum? The mystery of experimental validation of apparently erroneous computational analyses of protein sequences. Genome Biol 2001, 2:RESEARCH0051.

12. A Bateman, E Birney, L Cerruti, R Durbin, L Etwiller, S R Eddy, S Griffiths-Jones, K L Howe, M Marshall, E L Sonnhammer: The Pfam protein families database. Nucleic Acids Res 2002, 30:276-280.

13. A J Gianotti, P A Tower, J Fi Sheley, P A Conte, C Spiro, A J Ferro, J H Fitchen, M K Riscoe: Selective killing of Klebsiella pneumoniae by 5-trifluoromethylthioribose. Chemotherapeutic exploitation of the enzyme 5-methylthioribose kinase. J Biol Chem 1990, 265;831-837,

14. E S Purfine, R H Abeles: Intermediates in the conversion of 5′-S-methylthioadenosine to methionine in Klebsiella pneumoniae. J Biol Chem 1988, 263:9598-9606.

15. J W Wray, R H Abeles: The methionine salvage pathway in Klebsiella pneumoniae and rat liver. Identification and characterization of two novel diosygenases. J Biol Chem X995, 270:314?-3153.

16. S W Myers, R H Abeles: Conversion of 5-S-methyl-5-thio-D-ribose to methionine in Klebsiella pneumoniae. Stable isotope incorporation studies of the terminal enzymatic reactions in the pathway. J Biol Chem 1990, 265:16913-16921.

17. A Sekowska, A Danchin, J L Risler: Phylogeny of related functions: the case of polyamine biosynthetie enzymes. Microbiology 2000, 148:18151828.

18. G Bertani: Studies in lysogenesis I. The mode of phage liberation by lysogenic Escherichia coli. J Bacteriol 1951, 62:293-300.

19. F Schlenk, C R Zydek-Cwick, J L Dainko: 5′-Methylthioadenosine and related compounds as precursors of S-adenosylmethionine in yeast. Biochimica et Biophysics Acta 1973, 320:357-362.

20. M Bouchu, S Large, M Steng, B Langlois, J- P Praly: An unprecedented access to trifluoromethylthiosugar derivatives from thiocyanate precursors upon treatment with trifluoromethylsilane. CarbohydrRes 1998, 314: 37-45.

21. J Sambrook, E Fritsch, T Maniatis: Molecular cloning: A laboratory manual., 2ed edn. Cold Spring Harbour, N.Y.: Cold Spring Harbour Laboratory Press; 1989.

22. F Kunst, G Rapoport: Salt stress in an environmental signal affecting degradative enzyme synthesis in bacillus subtilis. J Bacteriol 1995, 177:2403-2407.

23. C Saunders, B Schmidt, M Morot, L Thompson, M Guyer: Use of chromosomal integration in the establishment and expression of blaZ, a Staphylococcus aureus b-lactamase gene, in Bacillus subtilis. J Bacteriol 1984, 157:71$-726.

24. L Kim, A Mogk, W Schumann: A xylose-inducible Bacillus subtilis integration vector and its application. Gene 1996, 181:71-76.

25. M Perego: Integrational vectors for genetic manipulation in Bacillus subtilis. In: Bacillus subtilis and other Gram-positive Bacteria Edited by A L Sonenshein, J A Hoch, R LosicK. Washington, D.C.: American Society for Microbiology; 1993: 615-624.

26. E Murphy: Nucleotide sequence of a spectinomycin adenyltransferase AAD(9) determinant from Staphylococcus aureus and its relationship to AAD(3′)(9). Mol Gen Genet 1985, 200:33-39.

27. M Steinmetz, R Richter: Easy cloning of mini-Tn10 insertions from the Bacillus subtilis chromosome. J Bacteriol 1994, 176:1761-1763.

28. V Dartois, T Djavakhishvili, J A Hoch: KapB is a lipoprotein required for KinB signal transduction and activation of the phosphorelay to sporulation in Bacillus subtilis. Mol Microbiol 1997, 26:1097-1108.

29. C Piekielny, M Rosbash: mRNA splicing efficiency in yeast and contribution of non conserved sequences. Cell 1988, 41:119-126.

30. T Msadek, F Kunst, D Henner, A Klier, G Rapoport, R Dedonder: Signal transduction pathway controlling synthesis of a class of degradative enzymes in Bacillus subtilis: expression of the regulatory genes and analysis of mutations in degS and degU. J Bacteriol 1990, 1.72:824-834.

31. J Spizizen: Transformation of biochemically deficient strains of Bacillus subtilis by deoxyribonucleate. Proc Natl Acad Sci USA 1958, 44:1072-1078.

32. S Auger, W H Yuen, A Danchin, T Martin-Verstraete: The metIC operoxa involved in methionine biosynthesis in Bacillus subtilis is controlled by transcription antitermination. Microbiology 2002, 148:507-518.

TABLE 1
Phenotype of gene inactivation in the mtn region.

| Gene name | Strain | Growth on MTR as sole sulfur source |
|---|---|---|
| Wild type + O2^{a} | 168 | normal growth after four days |
| Wild type + O2^{a} | 168 | no growth after four days |
| mtnS | BSHP7010 | no growth |
| mtnK | BFS1850 | no growth |
| mtnU | BFS1851 | no growth (numerous revertants) |
| mtnV | BSHP7020 | normal growth |
| mtnW | BSHP7014 | no growth |
| mtnX | BFS1852 | normal growth |
| mtnY | BSHP7016 | no growth |
| mtnZ | BFS1853 | weak residual growth |

^{a}See Materials and methods; nitrate was used as an electron acceptor.

TABLE 2
Expression of mtn::lacZ transcriptional fusions.

| β-galactosidase Activity (U mg⁻¹ of protein)^{a} | |
|---|---|
| ED1 medium | ED1 medium |
| with sulfate | with methionine |

| Strain | exp^{b} | stat | exp | stat |
|---|---|---|---|---|
| BFS1851^{c} | 62 | 41 | 53 | 33 |
| BSHP7020 | 217 | 121 | 181 | 161 |
| BSHP7014 | 579 | 267 | 113 | 95 |
| BFS1852 | 442 | 251 | 108 | 92 |
| BSHP7016 | 294 | 139 | 61 | 47 |
| BFS1853 | 280 | 112 | 57 | 33 |

^{a}for the β-galactosidase activity assay the bacteria were grown in the ED minimal medium wiht either sulfate or methionine as sulfur source.
^{b}exp = exponential growth phase, stat = stationary growth phase.
^{c}BFS1851 = mtnU::lacZ, BSHP7020 = mtnV::lacZ, BSHP7014 = mtnW::lacZ, BFS1852 = mtnXL::lacZ, BSHP7016 = mtnY::lacZ, BFS1853 = mtnZ::lacZ.

TABLE 3
Bacterial strains and plasmids used in this study.

| | | Source or |
|---|---|---|
| Strain or plasmid | Genotype or description | reference |
| Strains | | |
| Escherichia coli | | |
| TG1 | K12 supE hsdΔ5 thi Δ (lac-proAB) | Laboratory |
| | F′ [traD36 proA + proB + lacI^{q} | collection |
| | lacZΔM15] | |
| XL1-BIue | K12 supE44 hsdR17 recA1 endA1 | Laboratory |
| | gyrA46 thi relA1 lac F′[proAB + lacI^{q} | collection |
| | lacZΔM15 Tn10(tet^{R})] | |
| Bacillus subtilis | | |
| 168 | trpC2 | [31] |
| BSIP1143 | trpC2metI::spc | [32] |
| BSHP7010 | trpC2mtnS..spc | [6] |
| BFS1850 | trpC2 mtnK::lacZ | Functional |
| | | analysis |
| | | project^{a} [6] |
| BFS1851 | trpC2 mtnU::lacZ | Functional |
| | | analysis |
| | | project^{a} [6] |
| BFS1852 | trpC2 mtnX::lacZ | Functional |
| | | analysis |
| | | project^{a} |
| BFS1853 | trpC2 mtnZ::lacZ | Functional |
| | | analysis |
| | | project^{a} |
| BSHP7014 | trpC2 mtnW:.lacZ | This work |
| | amyE::(pxylmtnXYZ) | |
| BSHP7015 | trpC2 amyE::(pxylmtnXYZ) | This work |
| BSHP7016 | trpC2 mtnY::lacZ | This work |
| BSHP7020 | trpC2 mtnV::lacZ | This work |
| BSHP7064 | trpC2 mtnW::Tn10 | This work |
| BSHP7065 | trpC2 mtnY::Tn10 | This work |
| Plasmids | | |
| pIC333 | mini-Tn10 delivery vector, Spc^{R}, Ery^{R} | [27] |
| pJM783 | cloning vector, Cm^{R}, Amp^{R} | [25] |
| pX | cloning vector, Cm^{R}, Amp^{R}pxyl | [24] |
| | promoter, amyE locus integration | |
| pHPP7011 | pJM mtnV::lacZ | This work |
| pHPP7014 | pJM mtnW::lacZ | This work |
| pHPP7014bis | pJM mtnW::lacZ (bla::spc^{b}) | This work |
| pHPP7015 | pX pxyl mtnXYZ | This work |
| pHPP7016 | pJM mtnY::lacZ | This work |

^{a}This strain has been constructed in the frame of the EC project for the functional characterization of the genome of B. subtilis in Europe.
^{b}spc is the spectinomycin resistance gene from Staphylococcus aureus.

1. A method of controlling cell multiplication, comprising interfering with several metabolic pathways simultaneously.

2. The method of claim 1, wherein at least two metabolic pathways are interfered with simultaneously.

3. The method of claim 1, wherein one of the metabolic pathways includes MtnW.

4. The method of claim 1, wherein one of the metabolic pathways includes MtnU.

5. A method of controlling cell multiplication, comprising interfering with several sulfur metabolic pathways simultaneously.

6. The method of claim 5, wherein at least two metabolic pathways are interfered with simultaneously.

7. The method of claim 5, wherein one of the metabolic pathways includes MtnW.

8. The method of claim 5, wherein one of the metabolic pathways includes MtnU.

9. A method of controlling cell multiplication, comprising interfering with the methylthioadenosine recycling pathway.

10. The method of claim 9, wherein MtnW is interfered with.

11. The method of claim 1, wherein MtnU is interfered with.

12. A method of controlling cell multiplication, comprising interfering with the methylthioadenosine recycling pathway.

13. The method of claim 1, wherein MtnW is interfered with.

14. The method of claim 1, wherein MtnU is interfered with.

15. A method of controlling cell multiplication, comprising interfering with the methylthioribose recycling pathway in plants.

16. The method of claim 1, wherein MtnW is interfered with.

17. The method of claim 1, wherein MtnU is interfered with.

18. A method of controlling cell multiplication, comprising interfering with the methylthioribose recycling pathway in Bacilli.

19. The method of claim 1, wherein MtnW is interfered with.

20. The method of claim 1, wherein MtnU is interfered with.

21. A method of identifying methylthioribose recycling enzymes as a drug target.

22. A method of identifying homologs of the MtnW and/or MtnU genes as drug targets.

23. A method of identifying homologs of the MtnW and/or MtnU genes in Bacillus subtilis as elements of methylthioribose metabolism.

24. A method of identifying Bacillus subtilis MtnW homologs as a specific step in methylthioribose metabolism.

25. A method of constructing Genetically Modified Organisms possessing all or part of the genes identified herein.

26. A method of constructing Genetically Modified Organisms lacking all or part of the genes identified herein.

27. A method as described in any of the preceding claims using all or part of sequences from Bacillus subtilis as templates for probe design for hybridization detection.

28. A method as described in any the preceding claims using all or part of protein sequences from Bacillus subtilis as templates for antibody design for immune detection.

29. An isolated and purified MtnW sequence.

30. An isolated and purified MtnU sequence.

31. The protein encoded by the MtnW gene.

32. The protein encoded by the MtnU gene.

33. The strains deposited at the CNCM under the accession number CNCM I-2858, CNCM I-2859, and CNCM I-2860.

34. A process of identifying compounds for activity against a bacilli infection by using at least one of the wild type genes of the bacilli as a target and a corresponding mutated gene or a recombinant bacteria carrying the wild type gene and a compound which may inhibit the activity of the genes.
